(19) United States
(12) Patent Application Publication
(10) Pub. No.: US 2026/0060729 A1
(43) Pub. Date: Mar. 5, 2026

(54) APPARATUS, SYSTEM AND METHOD FOR FIXATION OF GREATER TROCHANTER OF FEMUR

(71) Applicant: OrthoTangent LLC, Omaha, NE (US)

(72) Inventor: BRYAN D. BREDTHAUER, Omaha, NE (US)

(21) Appl. No.: 19/313,347

(22) Filed: Aug. 28, 2025

Related U.S. Application Data
(60) Provisional application No. 63/688,184, filed on Aug. 28, 2024.
(60) Provisional application No. 63/740,193, filed on Dec. 30, 2024.

(51) Int. Cl. A61B 17/80; A61B 17/17; A61B 17/56; A61B 17/82; A61B 17/86

(52) U.S. Cl. CPC A61B 17/8061; A61B 17/1725; A61B 17/1742; A61B 17/82; A61B 17/8625; A61B 17/864; A61B 2017/564

(57) ABSTRACT

A surgical implant device and method of using the same for fixation of the greater trochanter is provided. The surgical implant device comprises a plate defining a flat portion and a bulbous portion each defining pre-drilled holes configured to provide a drill guide for insertion of a plurality of screws and/or cables to hold the surgical implant device in the desired location. The device is configured to provide stable fixation of the greater trochanter while mitigating cable degradation and soft tissue damage/irritation present in legacy surgical techniques.

### CROSS-REFERENCE TO RELATED APPLICATIONS

This application claims priority pursuant to 35 U.S.C. 119(e) to co-pending U.S. Provisional Patent Application Ser. No. 63/688,184, filed Aug. 28, 2024, and co-pending U.S. Provisional Patent Application Ser. No. 63/740,193, filed Dec. 30, 2024, the entire disclosures of which are incorporated herein by reference.

### FIELD OF THE INVENTION

The present inventive concept relates generally to surgical implants and methods. More specifically, the present inventive concept relates to a surgical implant device and method for fixation of the greater trochanter of the femur in a human.

### BACKGROUND

The need for fixation of the greater trochanter (sometimes referred to herein as simply “the trochanter”) of the femur in a human adult may arise from a traumatic fracture, from the iatrogenic fracture during hip replacement or other surgery or from an extended trochanteric osteotomy of the femur performed during revision surgery.

The challenges and frequent failure of fixation of the trochanter relate to the nature of the bone and the forces exerted by the abductor musculature. The greater trochanter is primarily cancellous bone which even in its native state is soft and difficult to stabilize with plates and screws that work well in cortical bone. In the arthroplasty setting the bone is often osteoporotic and less able to be contained by existing methods. The trochanteric fragment tends to displace proximal and anterior due to the pull of the gluteus medius and gluteus minimis.

The legacy surgical technique for trochanteric fixation typically involve placement of steel wires through and around the trochanteric bone, attaching it to the shaft of the femur. This method often leads to the fatigue fracture of the wires and loss of fixation.

Various clamps and grips applied to the external surface of the trochanteric fragment have been used over the last 50 years. An early example is found in U.S. Pat. No. 3,824,995 (the “'995 Patent”) from 1974, the entire disclosure of which is incorporated herein by reference. A more modern example of a similar design of the '995 Patent is disclosed in U.S. Pat. No. 6,338,734 (the “'734 Patent”) the entire disclosure of which is incorporated herein by reference, that in various forms is still in common usage. Designs like the '995 Patent and the '734 Patent utilize a metal plate extending over the lateral aspect of the trochanter that has short spikes that extend into the soft bone of the trochanter. Nevertheless, these designs fail to provide adequate fixation between the metal plate and the greater trochanter without utilizing wires and cables similar to the legacy technique, thereby posing similar limitations. In addition, the prominence of the metal plate over the lateral aspect of the trochanter commonly leads to pain due to soft tissue irritation and impingement. Even if the bone heals, the patient commonly has pain due to the presence of the implant.

Another concept for trochanteric fixation involves proximally extending arms or a loop of a laterally applied plate, with screws placed into the trochanter. While this design eliminates the problems relating to the lateral prominence of the plate, fixation in poor quality bone is lacking. An example of this concept is illustrated in U.S. Pat. No. 8,906,072 (the “'072 Patent”), the entire disclosure of which is incorporated herein by reference.

In the context of intertrochanteric fracture, various devices have been used which provide a plate applied over the lateral side of the femoral shaft which provide a drill guide for extending screws into the femoral head. An example of such a device is disclosed in U.S. Pat. No. 2,628,614, the entire disclosure of which is incorporated herein by reference. However, such devices or other similar designs have not found prominent use in treating fractures of the greater trochanter due to such devices not providing any fixation of the greater trochanter.

### SUMMARY

The inventive concept provides an apparatus/device, system and method for fixation of the greater trochanter of the femur in a human, including a human adult. The surgical implant device of some embodiments of the inventive concept includes a generally flat plate that includes a bulbous portion generally shaped to follow the curvature of the greater trochanter as it protrudes from the femur. In some embodiments, the generally bulbous portion of the plate includes pre-formed/pre-drilled holes. In some embodiments, the preformed holes function as a guide for drilling holes into the trochanter for placement of screws through the fractured fragment of the trochanter. Once holes are drilled, the screws are advanced through the plate and into the fragment. In some embodiments, the preformed holes are threaded for receiving mating threads of the screws. In some embodiments the generally flat portion of the plate includes predrilled holes through which screws are advanced to fix the plate to the femur. In some embodiments, a cable is used to fix the plate to the femur.

The advantages of embodiments of the device of the inventive concept are several. In some embodiments, the plate of the device sits distal to the trochanteric crest and is less likely to cause soft tissue irritation. Similar plate placement has been used for decades for fixation of intertrochanteric fractures and is well tolerated.

The use of cannulated screws through the trochanter in some embodiments allows for fixation of osteoporotic bone and resistance of the anterior pull of the abductors. In some embodiments metal or polymer cable is capable of being passed through the screws and over the bone of the trochanter, counteracting the proximal pull of the muscles.

In the case of fixation of an extended trochanteric osteotomy, the plate of some embodiments of the inventive concept is capable of being used to span the length of the osteotomy fragment, which eliminates the tendency of the fragment to migrate proximally and also decreases the risk of a cerclage cable around the femoral shaft causing a fracture of the trochanter.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

Embodiments of the present invention are described in detail below with reference to the attached drawing figures, wherein:

FIG. 1 shows an anterior-posterior view of a femur having a fracture of the greater trochanter with a device of an embodiment of the inventive concept implanted.

FIG. 2 shows a side elevation view of the femur and the implant device of FIG. 1 of the inventive concept.

FIG. 3A shows an embodiment of a single-threaded screw used for fixing the plate to the trochanter and/or femur.

FIG. 3B shows a first embodiment of a multi-threaded screw used for fixing the plate to the trochanter and/or femur.

FIG. 3C shows a second embodiment of a multi-threaded screw used for fixing the plate to the trochanter and/or femur.

FIG. 4 shows another embodiment of the inventive concept similar to that in FIGS. 1 and 2 that includes a trochanter cable pulled through a cannulated screw.

FIG. 5 shows another embodiment of the inventive concept in which an implant of the inventive concept is used for stabilization of an extended trochanteric osteotomy fragment.

FIG. 6A provides partial rear and side elevation views of the top of embodiments of a plate of the present invention in which holes in a bulbous portion provide for a variable angle drill guide.

FIG. 6B show partial views of the tops of plates of various other embodiments of the inventive concept wherein the plates include straight and angled configurations.

FIG. 7 provides a cross-sectional view of an embodiment of a cannulated screw of the present invention.

FIG. 8 provides a transparent view of embodiments of a cannulated screw of the present invention with and without a cable.

FIGS. 9A and 9B provides top and side views, respectively, of embodiments of an eyelet screw of the present invention.

FIG. 10 shows and anterior-posterior view of a femur having a fracture of the greater trochanter with a device of an embodiment of the inventive concept implanted.

FIG. 11 shows a side elevation view of the femur and the implant device of FIG. 10 of the inventive concept.

The drawing figures do not limit the present invention to the specific embodiments disclosed and described herein. The drawings are not necessarily to scale, emphasis instead being placed upon illustrating the principles of the invention.

### DETAILED DESCRIPTION

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

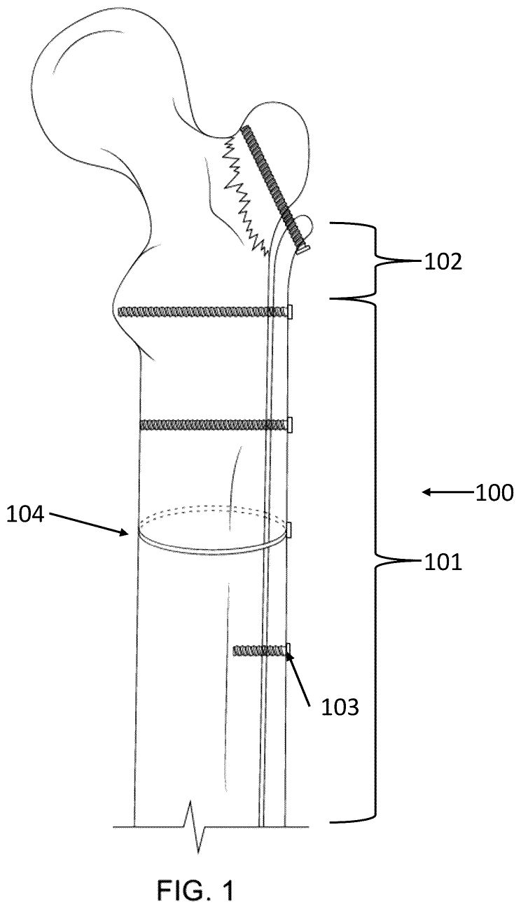

Referring to FIG. 1, an embodiment of the surgical implant device of the present invention is shown comprising a plate 100 configured for fixation of the greater trochanter of the femur in a human adult. The surgical implant device and/or plate of the present invention is movable between a fixed and unfixed configuration, wherein moving the device and/or plate from the unfixed configuration to the fixed configuration comprises fixedly coupling the plate 100 to the femur such that the greater trochanter is substantially fixed in a stable position. The plate 100 comprises a flat portion 101 configured to be positioned on the lateral side of the femoral shaft and fixedly coupled thereto when the device is in the fixed configuration.

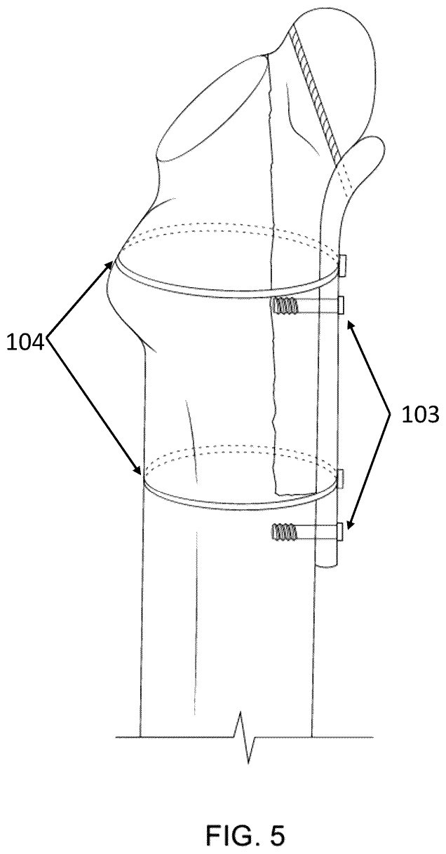

In some preferred embodiments, the flat portion 101 is of a sufficient length such that at least part of the flat portion 101 is fixedly coupled to a stable location on the femoral shaft (i.e. a location on the femur which is not fractured and/or part of a trochanteric osteotomy fragment) when the implant device and/or plate is in the fixed configuration. In some such preferred embodiments, such as the embodiment shown in FIG. 1, the length of the flat portion 101 substantially corresponds to the length of the femoral shaft. In some such preferred embodiments, such as the embodiment shown in FIG. 5, the flat portion 101 is of a sufficient length to extend past an extended trochanteric osteotomy fragment such that the plate 100 is capable of being fixedly coupled to a stable location on the femoral shaft, thereby eliminating the tendency of the osteotomy fragment to migrate away from its fixed position and also decreasing the risk of trochanteric fracture which may be influenced by the use of a cerclage cable 104 around the femoral shaft.

The plate 100 further comprises a bulbous portion 102 extending upwardly and away from the flat portion 101. In some embodiments, the bulbous portion defines a curved profile and the curvature of the bulbous portion 102 is configured such that the plate 100 defines a contour consistent with the contour of the greater trochanter as it protrudes from the femur. In the embodiment shown, the plate 100 is configured such that, when the device is in the fixed configuration, the bulbous portion 102 is positioned below the greater trochanter. Additionally, the length of bulbous portion 102 in the embodiment shown is configured such that, when the device is in the fixed configuration, it extends laterally from the femur less than that of the greater trochanter. The combination of the bulbous portion 102 being positioned below and not extending laterally past or over the greater trochanter mitigates potential soft tissue damage/irritation.

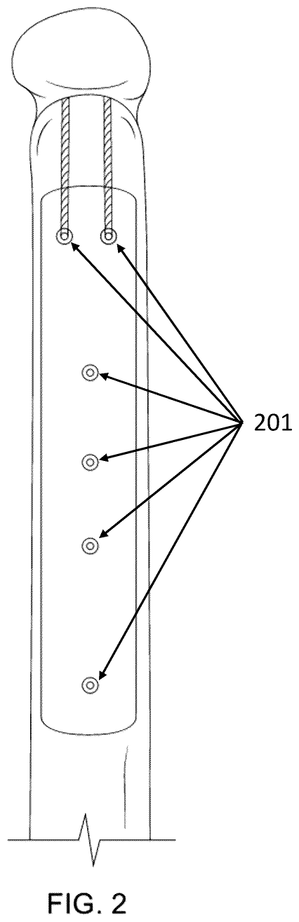

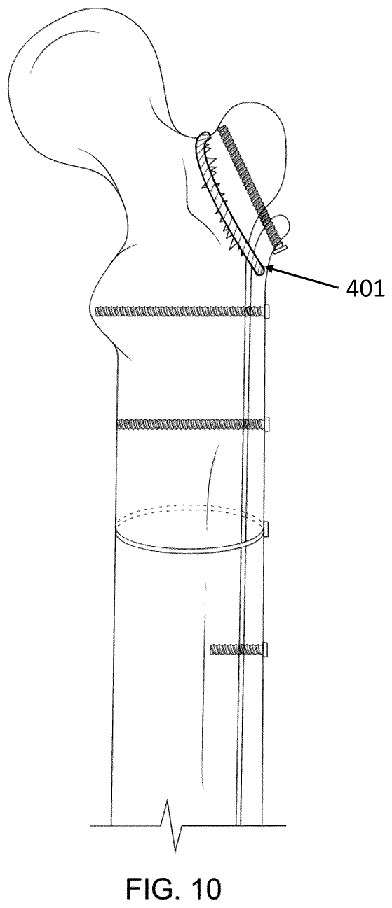

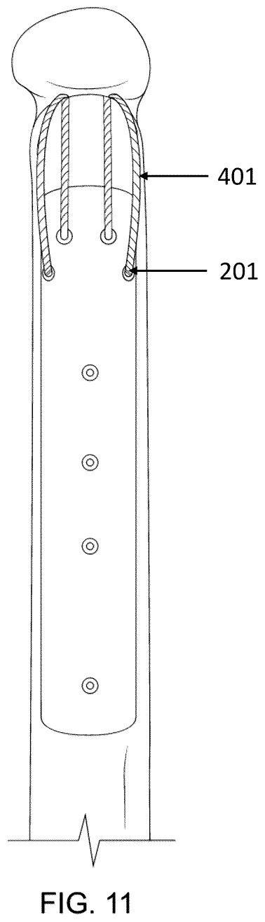

Referring to FIG. 2, the plate 100 defines a plurality of pre-drilled holes 201 extending through a front and/or side surface of the plate each configured to receive a corresponding fixation device such as screws and/or cable. In some embodiments of the implant device wherein the fixation devices are screws, the pre-drilled holes 201 are configured to provide a drill guide to ensure proper positioning of holes drilled into the bone and/or the corresponding screws as they are advanced into the bone. The plurality of pre-drilled holes 201 each define threading to secure the corresponding fixation devices in the plate 100 when the implant device is in the fixed configuration. The pre-drilled holes 201 includes at least one or more bulbous portion holes and/or one or more flat portion holes located in the bulbous and flat portions of plate 100, respectively. In some embodiments, such as those shown in FIGS. 1 and 2, the plate 100 defines two bulbous portion holes through the front surface of the plate and four flat portion holes through the front surface of the plate. In some embodiments, such as those shown in FIGS. 10 and 11, the plate 100 defines three bulbous portion holes, two through the front surface of the plate and one through a side surface of the plate, and four flat portion holes through the front surface of the plate. In other various embodiments, there are a variety of combinations of pre-drilled holes 201 in the flat portion, the bulbous portion, or both, which allow for stable fixation of plate 100 as herein described.

Due to the curved profile of the bulbous portion, the bulbous portion holes in some embodiments define a drill guide that defines an angle of insertion relative to the flat portion of the plate. The bulbous portion holes are, thereby, configured in some embodiments such that they define a drill guide to drill a hole into the bone in a proper location to receive one or more screws into the greater trochanter, thereby substantially fixing the greater trochanter in a stable position when the implant device is in the fixed configuration. In some embodiments, the angle of insertion is of a sufficient degree to direct the drill guide created by the bulbous portion holes into the greater trochanter. In some such embodiments, the angle of insertion is 165-170° relative to the flat portion of the plate.

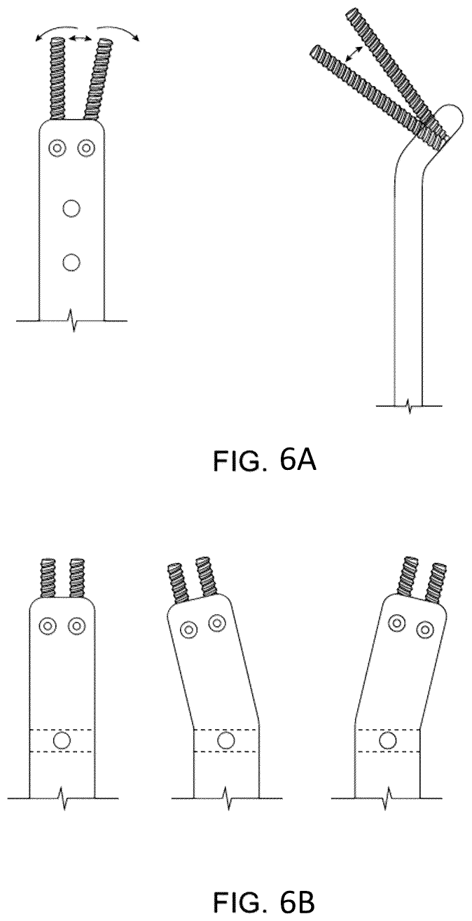

Referring to FIG. 6a, in some embodiments, the bulbous portion holes are configured such that the angle of insertion relative to the flat portion of the plate is variable, thereby making the bulbous portion holes define a variable angle drill guide configured to allow a physician to drill holes in a suitable position on the greater trochanter without requiring those holes to be perfectly in line with the bulbous portion holes. In some such embodiments, the drill guide defined by the bulbous portion holes is configured to be adaptable to the anatomy of a particular patient. In some embodiments, the variable angle is within a determined range of angles. In some embodiments, the variable angle drill guide is enabled by the bulbous portion holes defining threading configured to engage with the screws at a variety of different angles. Some of such embodiments use variable angle threaded holes such as that disclosed in U.S. Pat. No. 8,876,873, the entire disclosure of which is incorporated herein by reference. It will be appreciated that various embodiments of the inventive concept utilize fixed angle and variable angle hole technologies now known and hereafter developed.

Referring to FIG. 6b, in various embodiments the of the present invention, the plate 100 is configured such that there exists a plate angle between the bulbous portion and flat portions of the plate when the plate 100 is viewed from the back of the plate 100. In some embodiments, the plate 100 adopts a straight configuration, wherein the bulbous portion of the plate is generally in line with the flat portion of the plate such that the plate angle is at or substantially near 180°. In other embodiments, the plate 100 adopts an angled configuration wherein the bulbous portion is not in line with the flat portion of the plate and is angled to the right or left such that the plate angle is less or greater than 180°. The use of a plate 100 that adopts an angled configuration allows the implant device of the present invention to accommodate differing patient anatomy.

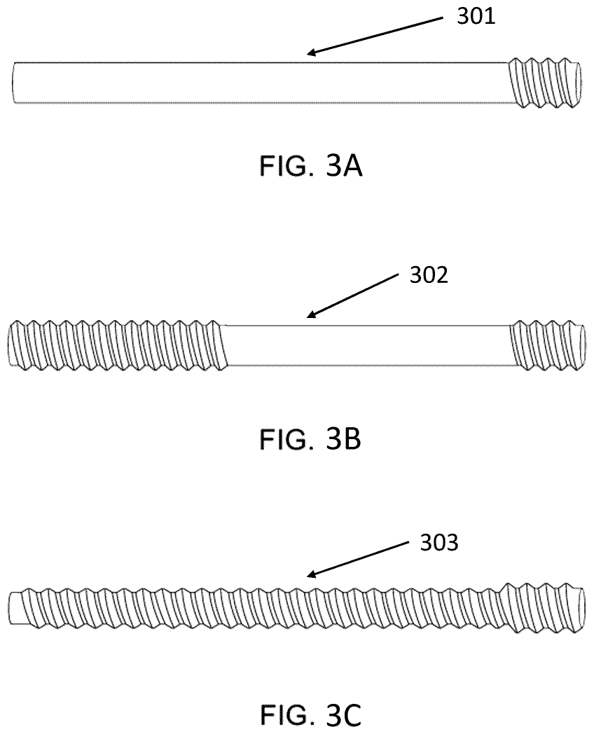

Referring to FIG. 3, various embodiments of the present invention utilize a variety of different screws as the fixation means to secure the surgical implant device in the fixed configuration. In some such embodiments, the surgical implant device utilizes a single threaded screw 301 which defines one segment of threading corresponding to the threading of the pre-drilled holes 201. In some embodiments, such single threaded screws are beneficial for use in locations of the plate which require less fixation. In other such embodiments, the surgical implant device utilizes a multi-threaded screw 302 and/or 303 which defines a first segment of threading corresponding to the threading of the pre-drilled holes 201 and a second segment of threading which is advanced into the bone to assist in securing the surgical implant device in the fixed configuration. In some such embodiments, such as those shown by the multi-threaded screws 302 and 303, the second segment of threading is of a smaller diameter than the first segment of threading so as to allow the second segment of threading to advance into the bone through the pre-drilled holes 201 prior to the first segment of threading securing the multi-threaded screw in place in the threads of the pre-drilled holes 201. In some embodiments, such as the multi-threaded screw 302, the first and second segments of threading are separated by a gap on the shaft of the screw to allow for the second segment of threading to define a greater pitch to apply variable compression to the bone in which it is inserted. In other embodiments, such as the multi-threaded screw 303, the first and second segments of threading are continuous along the shaft of the screw.

Referring back to FIG. 1, embodiments of the present invention utilize screws of different lengths configured to anchor into the bone at a desired depth as a fixation means. In some embodiments, the surgical implant device of the present invention utilizes screws which span the width of the femoral shaft when the implant device is in the fixed configuration. In other embodiments, a short locking screw 103 is utilized which is configured to anchor in the femoral cortex. In some embodiments, a short locking screw 103 is beneficial for use in patients with a hip prosthesis to avoid anchoring the screw at a depth which may contact the femoral stem of the hip prosthesis.

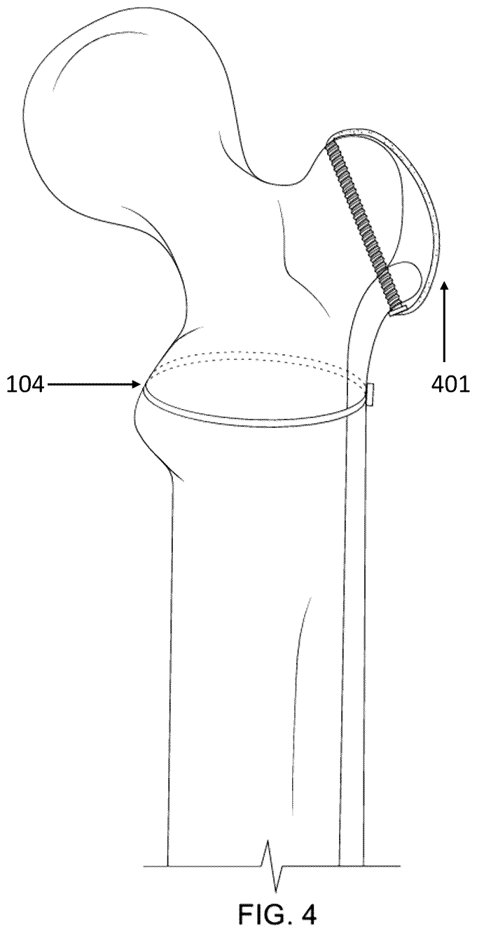

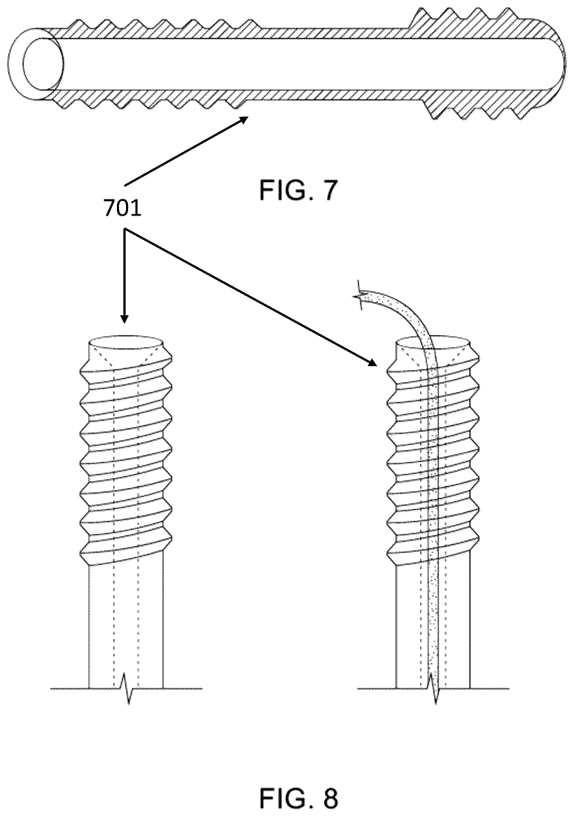

Referring to FIGS. 4, 7, and 8, in some embodiments the surgical implant device utilizes one or more cannulated screw 701 through one or more corresponding bulbous holes. The one or more cannulated screws 701 include a tunnel through the length of the screw, thereby allowing one or more trochanter cable 401 to be passed through the cannulated screws 701 and around the greater trochanter to secure the plate 100 to the greater trochanter when the device is in the fixed configuration. In some embodiments which utilize a cannulated screw 701, the ends of the tunnel through the cannulated screw 701 are beveled and/or polished to reduce the chance of abrasion of the cable that can be caused by sharp edges. Referring to FIGS. 10 and 11, in some embodiments, the plate 100 defines one or more pre-drilled holes 201 extending through a side-surface of the plate in the bulbous portion and configured to receive one or more corresponding trochanter cables 401. In some such embodiments, the edges of the pre-drilled hole 201 extending through a side-surface of the plate in the bulbous portion are beveled and/or polished to reduce the chance of abrasion of the cable that can be caused by sharp edges. The use of one or more cannulated screws 701 and/or one or more corresponding trochanter cables 401 to secure the implant device in some embodiments further secures the greater trochanter in place against the pull of abductor muscles. The use of one or more cannulated screws 701 and one or more corresponding trochanter cables 401 to secure the implant device in some embodiments also allows for the fixation of osteoporotic bone.

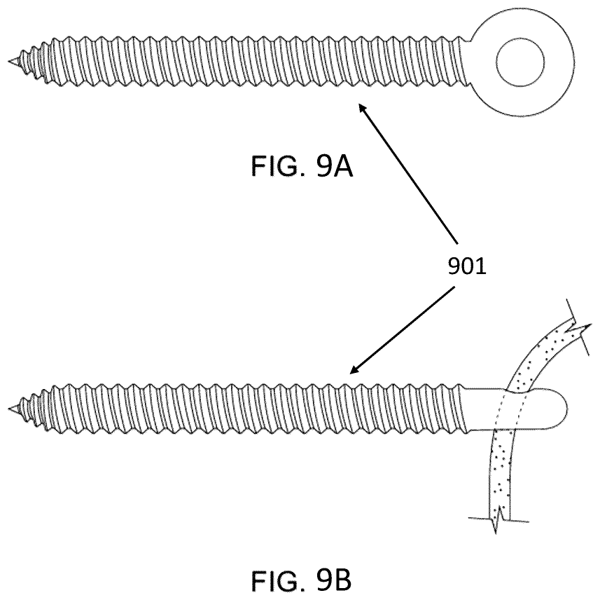

Referring to FIGS. 1, 4, 5, and 9, in some embodiments, the surgical implant device is secured in the fixed configuration utilizing one or more cerclage cables 104 as a fixation means. In some embodiments, the one or more cerclage cables are configured to be secured in one or more corresponding pre-drilled holes 201 in the flat portion 101 of plate 100. In other embodiments, the one or more cerclage cables comprise a head comprised of a soft metal, thereby allowing a physician to crimp/compress the metal of the head when the cerclage cable is in proper position, thereby holding the cerclage cable in place. In some embodiments, one or more eyelet screws 901 are utilized to secure the one or more cerclage cables 104 in the one or more corresponding pre-drilled holes 201 by threading the cerclage cable through an eyelet located on the non-working end of the eyelet screw 901. The one or more cerclage cables 104 are configured to wrap around the femoral shaft, thereby securing the implant device in the fixed configuration. The use of one or more cerclage cables 104 in some embodiments is beneficial for use in patients who have a hip prosthesis to provide a means of securing the implant device in the fixed configuration without anchoring any screw in any portion of the hip prosthesis.

In a preferred embodiment of the surgical implant device, the plate 100 is comprised of a titanium, ferrous or non-ferrous alloy material. In a preferred embodiment of the surgical implant device, screws made of a titanium, ferrous or non-ferrous material are utilized. In other various embodiments of the surgical implant device, the plate 100 and/or screws are comprised of a variety of biocompatible materials which are sufficiently rigid to achieve the functions described herein, such as stainless steel, cobalt-based alloys, titanium, carbon fiber, non-metallic alloys, etc., as such materials are known in the art.

The present invention also includes a method of fixation of the greater trochanter of a patient using the surgical implant device as described herein. The method comprises moving the surgical implant device of the present invention from the unfixed configuration to the fixed configuration, wherein moving the device from the unfixed configuration to the fixed configuration comprises fixedly coupling the plate 100 to the femur such that the greater trochanter is substantially fixed in a stable position. In some embodiments, fixedly coupling the plate 100 to the femur comprises securing one or more fixation means to the flat portion 101 of the plate 100 and the femoral shaft. In some such embodiments in which the fixation means includes one or more screws as described herein, securing the one or more fixation means to the flat portion 101 and the femoral shaft comprises utilizing the one or more pre-drilled holes 201 in the flat portion 101 as a drill guide for positioning one or more holes drilled into the femoral shaft configured to receive the one or more screws. In other such embodiments in which the fixation means comprises one or more cerclage cables 104, securing the one or more fixation means to the flat portion 101 and femoral comprises fixedly coupling the one or more cerclage cables to the flat portion 101 and wrapping the cerclage cable around the femoral shaft. It will be appreciated that in some embodiments of the inventive concept, the fixation means includes a combination of screws and/or cables.

In some embodiments, fixedly coupling the plate 100 to the femur comprises securing one or more fixation means to the bulbous portion 102 of the plate 100 and the greater trochanter. In some such embodiments in which the fixation means is one or more screws as described herein, securing the one or more fixation means to the bulbous portion 102 and the greater trochanter comprises utilizing the one or more pre-drilled holes 201 in the bulbous portion 102 as a drill guide for positioning one or more holes drilled into the greater trochanter configured to receive one or more screws. In some such embodiments in which the screws are cannulated screws, securing the one or more fixation means to the bulbous portion 102 and the greater trochanter comprises passing one or more trochanter cables 401 through the tunnel in the cannulated screw and wrapping it around the greater trochanter.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, various embodiments of the present technology include a variety of combinations and/or integrations of the embodiments described herein.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall within the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

What is claimed is:

1. A surgical implant device configured for fixation of the greater trochanter in a human comprising:
a plate which is movable between an unfixed configuration and a fixed configuration, wherein in said fixed configuration said plate is configured to be fixedly coupled to a femur such that the greater trochanter is substantially fixed in a stable position, said plate comprising:
a flat portion configured to be fixedly coupled to a lateral side of a femoral shaft of the femur when said plate is in said fixed configuration; and
a bulbous portion defining a curved profile, wherein said bulbous portion is configured to be positioned below the greater trochanter when said plate is in said fixed configuration,
wherein a contour of said curved profile of said bulbous portion substantially corresponds with a contour of the greater trochanter as it protrudes from the femur.

2. The surgical implant device of claim 1, wherein said bulbous portion defines a length such that, when said plate is in said fixed configuration, said bulbous portion extends laterally away from the femur less than a length of the greater trochanter.

3. The surgical implant device of claim 1, wherein said plate includes one or more pre-drilled holes in said flat portion and/or in said bulbous portion, and wherein at least one of said one or more pre-drilled holes is configured to receive a corresponding fixation device for securing said plate in said fixed configuration.

4. The surgical implant device of claim 3 wherein said fixation device comprises a screw.

5. The surgical implant device of claim 3 wherein said fixation device comprises a cerclage cable configured to wrap around the femoral shaft.

6. The surgical implant device of claim 3 wherein said fixation device comprises a cannulated screw.

7. The surgical implant device of claim 6 wherein said screw includes a tunnel extending through a length of said screw, said tunnel configured to receive a cable capable of wrapping around the greater trochanter.

8. The surgical implant device of claim 3 wherein at least one of said one or more pre-drilled holes includes a fixed-angle drill guide configured to secure said fixation device at a predetermined angle relative to said plate when said plate is in said fixed configuration.

9. The surgical implant device of claim 3 wherein at least one of said one or more pre-drilled holes defines a variable-angle drill guide configured to secure said fixation device within a range of angles relative to said plate when said plate is in said fixed configuration.

10. The surgical implant device of claim 1 wherein said bulbous portion of said plate extends away from said flat portion at an angle.

11. A surgical implant device configured for fixation of the greater trochanter of a femur in a human comprising:
a plate, said plate comprising:
a flat portion configured to be fixedly coupled to a lateral side of a femoral shaft of the femur, said flat portion including one or more flat portion holes; and
a bulbous portion configured to be positioned below the greater trochanter, said bulbous portion including one or more bulbous portion holes and a length that extends laterally away from the femur less than a length of the greater trochanter;
at least one fixation device configured to be received in one of the one or more flat portion holes or one of the one or more bulbous portion holes,
wherein said fixation device is configured to fixedly couple said plate to the femur.

12. The surgical implant device of claim 11, wherein at least one of said one or more flat portion holes or said one or more bulbous portion holes define a drill guide for positioning said fixation device in a desired location relative to the femur.

13. The surgical implant device of claim 11, wherein said flat portion of said plate includes a length substantially corresponding to a length of the femoral shaft.

14. The surgical implant device of claim 11, wherein said flat portion of said plate includes a length sufficient to extend past a fractured portion of the femur.

15. The surgical implant device of claim 11, wherein said fixation device comprises a screw that includes a length substantially corresponding to a width of the femoral shaft.

16. The surgical implant device of claim 11, wherein said fixation device comprises a short locking screw configured to anchor in a femoral cortex of the femur.

17. The surgical implant device of claim 11, wherein the fixation device comprises a cerclage cable configured to wrap around the femoral shaft.

18. A method of fixation of the greater trochanter of a femur in a human using a surgical implant device, the method comprising:
moving a surgical implant device from an unfixed configuration to a fixed configuration by fixedly coupling a plate of said surgical implant device to the femur such that the greater trochanter is substantially fixed in a stable position,
wherein said plate comprises a flat portion configured to be fixedly coupled to a lateral side of a femoral shaft of the femur when said plate is in said fixed configuration,
wherein said plate further comprises a bulbous portion including a curved profile and wherein said bulbous portion is configured to be positioned below the greater trochanter when said surgical implant device is in said fixed configuration.

19. The method of claim 18, wherein moving said surgical implant device from said unfixed configuration to said fixed configuration comprises using one or more pre-drilled holes in said plate as a drill guide for drilling a hole into the femur.

20. The method of claim 19, wherein moving said surgical implant device from said unfixed configuration to said fixed configuration further comprises securing a fixation device in at least one of said one or more pre-drilled holes of said plate, said fixation device configured to secure said surgical implant device in said fixed configuration.
